(19) United States
(12) Patent Application Publication
(10) Pub. No.: US 2005/0239735 A1
(43) Pub. Date: Oct. 27, 2005

(54) Enhancement of immune responses

(75) Inventors: Richard L. Miller, Maplewood, MN (US); Mark A. Tomai, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/027,037

(22) Filed: Dec. 30, 2004

Related U.S. Application Data
(60) Provisional application No. 60/533,143, filed on Dec. 30, 2003.

(51) Int. Cl. A61K 48/00; A61K 31/4745

(52) U.S. Cl. 514/44; 514/291; 514/292

(57) ABSTRACT

The present invention provides methods for enhancing the immune responses induced by IRM compounds. Generally, the methods include administering a cytokine receptor agonist or a cytokine inducer prior to administering an IRM compound to a cell population.

### CROSS-REFERENCE TO RELATED APPLICATION

This application claims priority to U.S. Provisional Patent Application Ser. No. 60/533,143, filed Dec. 30, 2003.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as toll-like receptors to induce selected cytokine biosynthesis. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153, 6,194,425, and 6,110,929) and more are still being discovered. Other IRMs have higher molecular weights, such as oligonucleotides, including CpGs (see, e.g., U.S. Pat. No. 6,1994,388).

In view of the great therapeutic potential for IRMs, and despite the important work that has already been done, there is a substantial ongoing need to expand their uses and therapeutic benefits.

### SUMMARY

It has been found that immune responses induced by certain small molecule IRMs can be enhanced by treating cells with a cytokine receptor agonist or a cytokine inducer prior to treatment with the IRM compound. Accordingly, the present invention provides a method of enhancing the immune response by treating cells with a cytokine receptor agonist or a cytokine inducer, followed by treating the cells with an IRM compound.

In some embodiments, the IRM compound may be an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In some embodiments, the cytokine receptor agonist may be a T_{H}1-promoting cytokine. In certain embodiments, the cytokine may be a Type I interferon (e.g., interferon-alpha, IFN-α). In other embodiments, the cytokine may be a Type II interferon (e.g., IFN-γ). In still other embodiments, the cytokine may be granulocyte-macrophage colony-stimulating factor (GM-CSF). In certain embodiments, the cytokine receptor agonist may be recombinant.

In some embodiments, the method may further include administering an antigen to the cells.

In another aspect, the present invention provides a method of enhancing the immune response by treating cells with a cytokine receptor agonist or a cytokine inducer followed by treating the cells with an IRM compound.

In another aspect, the present invention provides a method of treating a condition in a subject treatable by administering an immune response modifier by treating cells with a cytokine receptor agonist or a cytokine inducer and then treating the cells with an IRM compound. The cells may be treated in vivo or in vitro.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE INVENTION

The present invention relates to using certain cytokine receptor agonists or cytokine inducers to alter the immune response induced by IRM compounds. Accordingly, the invention provides a method for enhancing immune responses by treating cells with a cytokine receptor agonist or a cytokine inducer prior to treating the cells with an IRM compound.

Increasing a subject's immune response using a method of the invention can provide benefits in different ways. In some subjects, for example, the immune response induced by the administration of an IRM compound is lower than the immune response elicited in most other subjects. By pre-treating these subjects with a cytokine receptor agonist such as, for example, interferon-alpha (IFN-α), or a cytokine inducer, the immune response induced by the IRM compound can be enhanced. The method of the invention may allow these subjects to achieve the same immune response observed in most other subjects. Increasing a subject's immune response using the method of the invention also can increase the immune response against and, therefore, the efficacy of, for example, an immunological treatment such as a vaccine that otherwise possesses relatively low immunogenic potency. Also, methods of the present invention may allow one to achieve a desired level of immunological response to an antigen while using less of the antigen. This may be particularly desirable if the antigen is, for example, costly, rare, or otherwise difficult to obtain.

As used herein, the following terms shall have the indicated meanings:

“Agonist” and variations thereof refer to a compound that, in combination with a receptor (e.g., a cytokine receptor), can produce a cellular response (e.g., production of a cytokine). An agonist may be a ligand that directly binds to the receptor such as, for example, IFN-α, which can directly bind to the IFN-α receptor. Alternatively, an agonist may produce a cellular response indirectly by, for example, (a) forming a complex with another molecule that directly binds to the receptor, or (b) otherwise resulting in the modification of another compound so that the other compound directly binds to the receptor.

“Antigen” and variations thereof refer to any material capable of raising an immune response in a subject challenged with the material. In various embodiments, an antigen may raise a cell-mediated immune response, a humoral immune response, or both. Suitable antigens may be synthetic or occur naturally and, when they occur naturally, may be endogenous (e.g., a self-antigen) or exogenous. Suitable antigenic materials include but are not limited to peptides or polypeptides (including a nucleic acid, at least a portion of which encodes the peptide or polypeptide); lipids; glycolipids; polysaccharides; carbohydrates; polynucleotides; prions; live or inactivated bacteria, viruses, fungi, or parasites; and bacterial, viral, fungal, protozoal, tumor-derived, or organism-derived immunogens, toxins or toxoids.

“Cytokine inducer” and variations thereof refer to any compound that is capable of inducing the synthesis of a cytokine. Such compounds may be identified with respect one or more particular cytokines that are induced by the compound (e.g., interferon inducer). In some cases, the cytokine inducer may be a compound that binds to a receptor (e.g., a Toll-like receptor) and, through a cell signaling cascade, ultimately results in the synthesis and secretion of a cytokine.

“Cytokine receptor agonist” and variations thereof refer to a compound acts as an agonist, as defined above, for a cytokine receptor, thereby resulting in one or more biological effects associated with the cytokine. A cytokine receptor agonist may be the natural ligand for the cytokine receptor (i.e. a cytokine), but may in other cases be a synthetic (e.g., recombinant) form of the cytokine or a non-cytokine molecule (e.g. an antibody) capable of binding to the cytokine receptor and producing a cellular response.

Also, unless otherwise indicated, reference to a compound (whether an IRM compound, cytokine, cytokine receptor agonist, antigen, etc.) can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

Also herein, the recitations of numerical ranges by endpoints include all numbers subsumed within that range (e.g., 1 to 5 includes 1, 1.5, 2, 2.75, 3, 3.80, 4, 5, etc.).

Immune response modifiers (“IRMs”) include compounds that possess potent immunomodulating activity including but not limited to antiviral and antitumor activity. Certain IRM compounds modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}2 cytokines, such as IL-4 and IL-5. Additionally, some IRM compounds are said to suppress IL-1 and TNF (U.S. Pat. No. 6,518,265).

Certain IRM compounds are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; and U.S. patent Publication Nos. 2004/0091491; 2004/0147543; and 2004/0176367.

Additional examples of small molecule IRM compounds include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRM compounds include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRM compounds include biological molecules such as aminoalkyl glucosaminide phosphates (AGPs) and are described, for example, in U.S. Pat. Nos. 6,113,918; 6,303,347; 6,525,028; and 6,649,172.

The method of the invention includes administering a cytokine receptor agonist or a cytokine inducer to a cell population. In certain embodiments, the cytokine receptor agonist may be the natural ligand for the cytokine receptor. In some embodiments, the method of the invention includes administering a T_{H}1-promoting cytokine to a cell population. Suitable cytokines include, for example, a Type I interferon (e.g., IFN-α), a Type II interferon (e.g., IFN-γ), and granulocyte-macrophage colony-stimulating factor (GM-CSF).

In alternative embodiments, the cytokine receptor agonist may be a molecule other than the natural cytokine ligand for the cytokine receptor, but is still capable of inducing a cellular response from the cells of the cell population. In certain embodiments, a synthetic or recombinant cytokine such as, for example, recombinant IFN-α or recombinant IFN-γ may be administered to the cells. As another example, an agonistic antibody specific for a cytokine receptor (e.g., an anti-Type I interferon antibody) may be administered to the cells.

In some embodiments, the cytokine inducer may be an agonist of one or more TLRs. For example, double-stranded RNA (dsRNA) and a synthetic analog, poly(I:C), are known TLR3 agonists that can result in induction of interferon synthesis.

In various embodiments, the invention may alter a cell-mediated immune response, a humoral immune response, or both. In some embodiments, the invention may alter the response of specific immune cells including, but not limited to, B lymphocytes, T lymphocytes, dendritic cells, monocytes, macrophages, neutrophils, eosinophils, basophils, mast cells, or peripheral blood mononuclear cells (PBMCs). In one embodiment, the invention may be used to alter the response of, for example, PBMCs. In another embodiment, the method may be used to alter the response of dendritic cells. In another embodiment, the method may be used to alter the response of T lymphocytes. In another embodiment, the method may be used to alter the response of neutrophils. In yet another embodiment, the method may be used to alter the response of two or more types of immune cells.

The particular immune response altered by using the method of the invention may depend, at least in part, on the particular immune cells whose activity is altered as a result of using the method. For some types of immune cells (e.g., T lymphocytes), using the method of the invention can increase the level of cytokines or chemokines secreted by immune cells. As another example, the method of the invention may cause, for example, increased cell migration or enhanced antigen presenting function (e.g., dendritic cells). As yet another example, the method may induce an increase in the level of immunoglobulin (e.g., IgM, IgG, or IgA) produced and secreted by B lymphocytes. As yet another example, the method may induce a more pronounced decrease in certain cytokines such as, for example, IL-4, IL-5, and IL-13, which are known to aggravate certain atopic conditions.

In some embodiments, the immune response to a specific antigen may be enhanced. For example, a suitable antigen may be administered along with the cytokine receptor agonist and/or IRM compound to enhance the immune response directed at the administered antigen.

In one embodiment, the invention provides a method for enhancing the immune response induced by IRM compounds by pre-treating cells with a cytokine receptor agonist or a cytokine inducer. Generally, the method includes administering an IRM compound after administering a cytokine receptor agonist or a cytokine inducer. As used herein, “administering an IRM compound after administering a cytokine receptor agonist or cytokine inducer” refers to administering the cytokine receptor agonist (or cytokine inducer, as the case may be) and the IRM compound at temporally distinct times, as opposed to co-administration. For example, the cells may be treated with a cytokine receptor agonist or cytokine inducer for about 12 hours to about 24 hours, and then treated with an IRM compound, although the method of the invention may be practiced by treating the cells with cytokine receptor agonist or cytokine inducer for periods outside this range before treating the cells with IRM compound. In other embodiments, the IRM compound may be administered sooner than 12 hours after the cytokine receptor agonist or cytokine inducer is administered. For example, the cells may be treated with a cytokine receptor agonist or cytokine inducer and then treated with an IRM compound at least 15 minutes later. In another embodiment, the cells may be treated with IRM compound at least 30 minutes after being treated with cytokine receptor agonist or cytokine inducer. In another embodiment, the IRM compound may be administered at least four hours after the cytokine receptor agonist or cytokine inducer is administered. In another embodiment, the IRM compound may be administered up to about 36 hours after the cytokine receptor agonist or cytokine inducer is administered. In yet another embodiment, the IRM compound may be administered up to about 48 hours after the cytokine receptor agonist or cytokine inducer is administered.

In some embodiments, the method of the invention can be performed in vivo. For example, a subject may be treated with a cytokine receptor agonist or cytokine inducer and then treated with an IRM compound. In certain embodiments, the cytokine receptor agonist treatment (or cytokine inducer treatment, as the case may be) may be carried out by systemic or local administration. For example, a subject may be treated by administering a cytokine receptor agonist or cytokine inducer systemically, e.g., intravenously. Alternatively, a subject may be treated by administering a cytokine receptor or cytokine inducer agonist locally, (i.e., to a specific area of a subject).

The IRM compound may be administered in the same manner or in a different manner than that used to administer the cytokine receptor agonist or cytokine inducer. For example, the cytokine receptor agonist or cytokine inducer may be administered systemically and the IRM compound may be administered locally. In one particular embodiment, for example, recombinant IFN-α may be administered systemically, then an IRM compound may be administered locally such as, for example, topically.

In alternative embodiments, the method of the invention may be carried out in vitro. For example, cells may be isolated from a subject and then treated with a cytokine receptor agonist or cytokine inducer and then treated with an IRM compound. In some embodiments, the cells treated by the method of the invention may be administered to a subject. The subject may or may not be the original donor of the treated cells. For example, the cells may be isolated from a subject, treated in vitro according to the method of the invention, and then administered back to the subject. Alternatively, the cells may be collected from a donor, treated in vitro, and the treated cells may be administered to a subject.

Certain IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In one specific embodiment, the IRM compound is 4-amino-α,α,2-trimethyl-1H-imidazo[4,5-c]quinoline-1-ethanol. In another embodiment, the IRM compound is 1-(2-methylpropyl)-1H-imidazo[4,5-c]quinolin-4-amine. In another embodiment, the IRM compound is 4-amino-α,α-dimethyl-1H-imidazo[4,5-c]quinoline-1-ethanol. In another embodiment, the IRM compound is 4-amino-α,α-dimethyl-2-ethoxymethyl-1H-imidazo[4,5-c]quinolin-1-ethanol.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In some embodiments, the IRM compound may be a naphthyridine amine such as, for example, 2-methyl-1-(2-methylpropyl)-1H-imidazo[4,5-c][1,5]naphthyridin-4-amine or 1-(2-methylpropyl)-1H-imidazo[4,5-c][1,5]naphthyridin-4-amine.

In other embodiments the IRM compound may be a thiazoloquinoline amine such as, for example, 2-propylthiazolo[4,5-c]quinolin-4-amine.

In still other embodiments the IRM compound may be a sulfonamide substituted imidazoquinoline amine such as, for example, N-[4-(4-amino-2-ethyl-1H-imidazo[4,5-c]quinolin-1-yl)butyl]methanesulfonamide or N-{2-[4-amino-2-(ethoxymethyl)-1H-imidazo[4,5-c]quinolin-1-yl]-1,1-dimethylethyl}methanesulfonamide.

Suitable IRM compounds also may include the purine derivatives, imidazoquinoline amide derivatives, benzimidazole derivatives, adenine derivatives, and oligonucleotide sequences described above.

In some embodiments of the invention, the IRM compound may be a small molecule immune response modifier (e.g., molecular weight of less than about 1000 Daltons).

In some embodiments, the IRM compound may be a compound identified as an agonist of one or more TLRs. In some embodiments, the IRM compound can act as an agonist of one or more of TLR6, TLR7, or TLR8. The IRM may also in some cases be an agonist of TLR 9. In some embodiments, the IRM compound may be an agonist of TLR7 such as, for example, a TLR7-selective agonist. In other embodiments, the IRM compound may be a TLR8 agonist such as, for example, a TLR8-selective agonist. In still other embodiments, the IRM compound may be a TLR7/8 agonist.

As used herein, the term “TLR8-selective agonist” refers to any compound that acts as an agonist of TLR8, but does not act as an agonist of TLR7. A “TLR7-selective agonist” refers to a compound that acts as an agonist of TLR7, but does not act as an agonist of TLR8. A “TLR7/8 agonist” refers to a compound that acts as an agonist of both TLR7 and TLR8.

A TLR8-selective agonist or a TLR7-selective agonist may act as an agonist for the indicated TLR and one or more of TLR1, TLR2, TLR3, TLR4, TLR5, TLR6, TLR9, or TLR10. Accordingly, while “TLR8-selective agonist” may refer to a compound that acts as an agonist for TLR8 and for no other TLR, it may alternatively refer to a compound that acts as an agonist of TLR8 and, for example, TLR6. Similarly, “TLR7-selective agonist” may refer to a compound that acts as an agonist for TLR7 and for no other TLR, but it may alternatively refer to a compound that acts as an agonist of TLR7 and, for example, TLR6.

The TLR agonism for a particular compound may be assessed in any suitable manner. For example, assays for detecting TLR agonism of test compounds are described, for example, in U.S. patent Publication No. US 2004/0132079, and recombinant cell lines suitable for use in such assays are described, for example, in International Patent Publication No. WO04/053057.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

In some cases, practicing the method of the invention can shift the biological activity induced by an IRM compound to that of an IRM compound of somewhat different TLR agonism. Thus, the method may be used to broaden the spectrum of clinically effective IRM compounds that may be useful for treating a particular condition.

For example, one TLR7-mediated biological activity can include production of IFN-α, which may be beneficial for treating certain conditions such as, for example, a viral infection. On the other hand, a TLR8-mediated biological activity can include production of tumor necrosis factor (TNF), which may aggravate certain conditions such as, for example, inflammatory disease such as rheumatoid arthritis. A particular TLR7/8 agonist IRM compound may be identified as being well-suited for treating a viral infection, perhaps because of efficacy and/or the extent of TLR7-mediated biological activity induced by the compound, but also perhaps because of other desirable characteristics such as, for example, low toxicity, being easy to formulate and deliver (formulability), cost, stability (e.g., shelf-life), bio-availability, metabolic half-life, etc. However, if administered to a subject having rheumatoid arthritis, the TLR8-mediated biological activity (TNF production) induced by the TLR7/8 agonist IRM compound may aggravate the rheumatoid arthritis to an extent that may prevent consideration of the TLR7/8 agonist IRM compound as a treatment for a viral infection in a patient that also has rheumatoid arthritis.

Practicing the present invention may allow such a subject to enjoy the benefits of treating one condition (e.g., the viral infection) with the TLR7/8 agonist IRM compound without aggravating the second condition (e.g., rheumatoid arthritis) to an intolerable extent. By pre-treating the subject with a cytokine receptor agonist before administering the TLR7/8 agonist IRM compound, sufficient TLR7-mediated biological activity may be induced by the TLR7/8 agonist IRM compound to provide treatment for the viral infection, while the TLR8-mediated biological activity induced by the TLR7/8 agonist IRM compound may be limited to acceptable levels—in some cases, even fully eliminating the TLR8-mediated biological activity. Thus, in the example above, pre-treating the subject with a cytokine receptor agonist before administering the TLR7/8 agonist may induce sufficient IFN-α to treat the a viral infection and reduce the amount of TNF induced by the TLR7/8 agonist IRM compound sufficiently so that the treatment of the a viral infection may proceed while limiting—or even eliminating—aggravation of the rheumatoid arthritis that would otherwise result from administering the TLR7/8 agonist IRM compound.

Each of the IRM compound and the cytokine receptor agonist (or cytokine inducer, as the case may be) may be provided in any formulation suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and International Patent Publication No. WO 03/045391. The IRM compound may be provided in any suitable form including but not limited to a solution, a suspension, an emulsion, or any form of mixture. The IRM compound may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including but not limited to adjuvants, skin penetration enhancers, colorants, fragrances, flavorings, moisturizers, thickeners, and the like.

An amount of an IRM compound effective for increasing a subject's immune response is an amount sufficient to induce or increase at least one biological activity associated with increasing an immune response such as, for example, the biological activities described above. The precise amount of MM compound for increasing a subject's immune response will vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, and the species to which the formulation is being administered. Accordingly, it is not practical to set forth generally the amount that constitutes an amount of IRM compound effective for increasing a subject's immune response for all possible applications. Those of ordinary skill in the art, however, can readily determine the appropriate amount with due consideration of such factors.

In some embodiments, the methods of the present invention include administering sufficient IRM compound to provide a dose of, for example, from about 100 ng/kg to about 50 mg/kg to the subject, although in some embodiments the methods may be performed by administering IRM compound in concentrations outside this range. In some of these embodiments, the method includes administering sufficient IRM compound to provide a dose of from about 10 μg/kg to about 5 mg/kg to the subject, for example, a dose of from about 100 μg/kg to about 1 mg/kg.

In some embodiments of the invention, the IRM compound may be administered once, although in some embodiments the invention may be practiced by administering the IRM compound more than once.

The cytokine receptor agonist or cytokine inducer may be provided in any suitable form including but not limited to a solution, a suspension, an emulsion, or any form of mixture. The cytokine receptor agonist or cytokine inducer may be delivered in formulation with any pharmaceutically acceptable excipient, carrier, or vehicle. The cytokine receptor agonist or cytokine inducer may be administered by any suitable route such as, for example, by subcutaneous, intravenous, transdermal, or transmucosal administration.

An amount of a cytokine receptor agonist or cytokine inducer effective for increasing a subject's immune response is an amount sufficient to induce or increase at least one biological activity associated with increasing an immune response such as, for example, the biological activities described above. The precise amount of cytokine receptor agonist or cytokine inducer for increasing a subject's immune response will vary according to factors known in the art including but not limited to the physical and chemical nature of the cytokine receptor agonist or cytokine inducer, the nature of the carrier, the intended dosing regimen, the state of the subject's immune system (e.g., suppressed, compromised, stimulated), the method of administering the cytokine receptor agonist or cytokine inducer, and the species to which the formulation is being administered. Accordingly, it is not practical to set forth generally the amount that constitutes an amount of cytokine receptor agonist or cytokine inducer effective for increasing a subject's immune response for all possible applications. Those of ordinary skill in the art, however, can readily determine the appropriate amount with due consideration of such factors.

In some embodiments, a sufficient amount of cytokine receptor agonist or cytokine inducer can be an amount necessary to attain a serum concentration of, for example, from about 1 pg/mL to about 1000 ng/mL to the subject, although in some embodiments the invention may be practiced by administering an amount of cytokine receptor agonist or cytokine inducer sufficient to attain a concentration outside this range. In some of these embodiments, one may practice the invention by administering sufficient cytokine receptor agonist or cytokine inducer to provide a dose of from about 5 pg/mL to about 50 ng/mL to the subject, for example, a dose of from about 10 pg/mL to about 100 pg/mL.

In some embodiments of the invention, the cytokine receptor agonist or cytokine inducer may be administered once, although in some embodiments one may practice the invention by administering the cytokine receptor agonist or cytokine inducer more than once.

Conditions for which the methods described herein may be used as treatments include, but are not limited to:
(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);
(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;
(c) other infectious diseases, such chlamydia, fungal diseases including but not limited to candidiasis, aspergillosis, histoplasmosis, cryptococcal meningitis, or parasitic diseases including but not limited to malaria, pneumocystis camii pneumonia, leishmaniasis, cryptosporidiosis, toxoplasmosis, and trypanosome infection;
(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;
(e) T_{H}2-mediated, atopic diseases, such as atopic dermatitis or eczema, eosinophilia, asthma, allergy, allergic rhinitis, and Ommen's syndrome;
(f) certain autoimmune diseases such as systemic lupus erythematosus, essential thrombocythaemia, multiple sclerosis, discoid lupus, alopecia areata; and (g) diseases associated with wound repair such as, for example, inhibition of keloid formation and other types of scarring (e.g., enhancing wound healing, including chronic wounds).

In certain embodiments, an immune response may be desired against a particular antigen such as, for example, an antigen associated with one of the conditions listed above. In such embodiments, the antigen (or at least an immunogenic epitope of the antigen) may be administered to the subject. The antigen may be co-administered with the cytokine receptor agonist or cytokine inducer, the IRM compound, or both. Alternatively, the antigen may be administered separately from the cytokine receptor agonist (or cytokine inducer, as the case may be) and/or IRM compound. When the antigen is administered separately, it may be administered before the cytokine receptor agonist or cytokine inducer is administered, after the IRM compound is administered, or in between the administration of the cytokine receptor agonist (or cytokine inducer) and the IRM compound.

An amount of antigen effective for use in certain embodiments of the invention is an amount sufficient to induce or increase at least one biological activity associated with increasing an immune response such as, for example, the biological activities described above. The precise amount of antigen for increasing a subject's immune response will vary according to factors known in the art including but not limited to the physical and chemical nature of the antigen, the nature of the carrier, the intended dosing regimen, the state of the subject's immune system (e.g., suppressed, compromised, stimulated), the potential enhancement of the immune response afforded by administration of the cytokine receptor agonist (or cytokine inducer) and the IRM compound, and the species to which the formulation is being administered. Accordingly, it is not practical to set forth generally the amount that constitutes an amount of antigen effective for increasing a subject's immune response for all possible applications. Those of ordinary skill in the art, however, can readily determine the appropriate amount with due consideration of such factors.

In some embodiments, the methods of the present invention include administering sufficient antigen to provide a dose of, for example, from about 100 ng/kg to about 50 mg/kg to the subject, although in some embodiments the methods may be performed by administering antigen in concentrations outside this range. In some of these embodiments, the method includes administering sufficient antigen to provide a dose of from about 10 μg/kg to about 5 mg/kg to the subject, for example, a dose of from about 100 μg/kg to about 1 mg/kg.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the examples are shown in Table 1.

TABLE 1

| Compound | Chemical Name | Reference |
|---|---|---|
| IRM1 | 4-amino-α,α,2-trimethyl-1H-imidazo[4,5- | U.S. Pat. No. 5,266,575, |
| | c]quinoline-1-ethanol | Example C1 |
| IRM2 | 1-(2-methylpropyl)-1H-imidazo[4,5-c]quinolin-4- | U.S. Pat. No. 4,689,338 |
| | amine | Example 99 |
| IRM3 | 4-amino-α,α-dimethyl-1H-imidazo[4,5- | U.S. Pat. No. 4,689,338 |
| | c]quinoline-1-ethanol | Example 189 |
| IRM4 | 4-amino-α,α-dimethyl-2-ethoxymethyl-1H- | U.S. Pat. No. 5,389,640 |
| | imidazo[4,5-c]quinolin-1-ethanol | Example 99 |
| IRM5 | 1-(2-methylpropyl)-1H-imidazo[4,5-c] | U.S. Pat. No. 6,194,425 |
| | [1,5]naphthyridin-4-amine | Example 32 |
| IRM6 | 2-methyl-1-(2-methylpropyl)-1H-imidazo[4,5-c] | U.S. Pat. No. 6,194,425 |
| | [1,5]naphthyridin-4-amine | Example 36 |
| IRM7 | 2-propylthiazolo[4,5-c]quinolin-4-amine | U.S. Pat. No. 6,110,929 |
| | | Example 12 |
| IRM8 | N-[4-(4-amino-2-ethyl-1H-imidazo[4,5-c]quinolin- | U.S. Pat. No. 6,677,349 |
| | 1-yl)butyl]methanesulfonamide | Example 236 |
| IRM9 | N-{2-[4-amino-2-(ethoxymethyl)-1H-imidazo[4,5- | U.S. Pat. No. 6,677,349 |
| | c]quinolin-1-yl]-1,1- | Example 268 |
| | dimethylethyl}methanesulfonamide | |

### Example 1

Adherent cells from isolated human peripheral blood mononuclear cells (PBMC) were treated as summarized in Table 1. The amount of Type I interferon (IFN) and tumor necrosis factor-α (TNF-α) produced by the cells in response to the treatment is also recorded in Table I.

TABLE 1

| IRM1 conc. | Pre-Treatment | IFN-α [U/mL] | TNF-α [pg/mL] |
|---|---|---|---|
| — | − | <1 | 41 |
| 3.0 μM | − | 5 | 3295 |
| 0.3 μM | − | 5 | 437 |
| — | + | 16 | 131 |
| 3.0 μM | + | 3,788 | 284 |
| 0.3 μM | + | 3,788 | 114 |

Briefly, adherent cells from human PBMC were treated with 1-100 U/mL recombinant IFN-α (Lee BioMolecular, San Diego, Calif.) for 24 hours, washed, and then treated with IRM (a concentrated solution of IRM in cell culture medium was added to reach the final concentration indicated in Table I) for an additional 24 hours. A separate group of control cells were treated only with IRM for 24 hours. Cell-free supernatants were then isolated from all of the cultures and analyzed for Type I IFN or TNF-α.

Type I IFN production was assayed using a virus neutralization bioassay using A549 human lung carcinoma cells challenged with encephalomyocarditis. The details of the bioassay method have been described by G. L. Brennan and L. H. Kronenberg in “Automated Bioassay of Interferons in Micro-test Plates”, Biotechniques, June/July, 78, 1983, incorporated herein by reference. Briefly stated the method is as follows: A549 cells are incubated with dilutions of samples or a standard interferon at 37° C. for 24 hours. The incubated cells are then infected with an inoculum of encephalomyocarditis virus. The infected cells are incubated for an additional 24 hours at 37° C. before evaluating for viral cytopathic effect. The viral cytopathic effect is quantified by staining with crystal violet followed by visual scoring of the plates. Results are expressed as IFN-α reference units/mL based on the value obtained for NIH Human Leukocyte IFN standard. TNF-α production was determined by ELISA (Biosource International, Inc., Camarillo, Calif.). The results are shown in Table 1.

### Example 2

Adherent cells are obtained and prepared as described in Example 1. The cells are pre-treated as described in Example 1 with either poly(I:C), recombinant IFN-γ, recombinant IFN-α, or receive no pre-treatment. The cells are washed as described in Example 1, then treated as described in Example 1 with either IRM1, IRM2, IRM3, IRM4, IRM5, IRM6, IRM7, IRM8, or IRM9 for 24 hours.

Cell free supernatants are isolated from all of the cultures and analyzed for Type I IFN or TNF-α. Results will show that, when pre-treated with a cytokine receptor agonist or cytokine inducer, the cells produce more Type I interferon and less TNF compared to untreated cells.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of enhancing an immune response, the method comprising:
treating a cell population with a cytokine receptor agonist or a cytokine inducer; and then
treating the cell population with an IRM compound.

2. The method of claim 1 wherein the IRM compound comprises an oligonucleotide sequence.

3. The method of claim 1 wherein the IRM compound comprises a purine derivative, an imidazoquinoline amide derivative, an imidazopyridine derivative, a benzimidazole derivative, or a derivative of a 4-aminopyrimidine fused to a five membered nitrogen containing heterocyclic ring.

4. The method of claim 1 wherein the IRM compound has a molecular weight of about 1000 Daltons or less.

5. The method of claim 1 wherein the IRM compound is a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

6. The method of claim 1 wherein the IRM compound is an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

7. The method of claim 1 wherein the IRM compound is a sulfonamide substituted imidazoquinoline amine.

8. The method of claim 1 wherein the IRM compound is a naphthyridine amine.

9. The method of claim 1 wherein the IRM compound is a thiazoloquinoline amine.

10. The method of claim 1 wherein the cytokine receptor agonist is a cytokine

11. The method of claim 1 wherein the cytokine receptor agonist is synthetic.

12. The method of claim 1 wherein the cytokine inducer is an agonist of a Toll-like receptor.

13. The method of claim 1 wherein the IRM compound is administered after the cytokine receptor agonist or cytokine inducer is administered.

14. The method of claim 1 wherein the IRM compound is administered at least 30 minutes after the cytokine receptor agonist or cytokine inducer is administered.

15. The method of claim 1 wherein the IRM compound is administered at least 4 hours after the cytokine receptor agonist or cytokine inducer is administered.

16. The method of claim 1 wherein the IRM compound is administered at least 24 hours after the cytokine receptor agonist or cytokine inducer is administered.

17. The method of claim 1, further comprising administering an antigen to the cell population.

18. A method of treating a condition in a subject treatable by administering an immune response modifier, the method comprising:
treating cells with a cytokine receptor agonist or a or cytokine inducer; and then
treating the cells with an immune response modifier.

19. The method of claim 18 wherein the cells are treated with the cytokine receptor agonist or cytokine inducer and the immune response modifier in vivo.

20. The method of claim 18 wherein the cells are treated with the cytokine receptor agonist or cytokine inducer and the immune response modifier in vitro.

21. The method of claim 20 wherein the treated cells are administered to a subject.

22. The method of claim 21 wherein the cells are collected from a donor that is not the subject.
